(19) United States
(12) Patent Application Publication
(10) Pub. No.: US 2022/0400945 A1
(43) Pub. Date: Dec. 22, 2022

(54) COLOR VISION DEFICIENCY BASED TEXT SUBSTITUTION

(71) Applicant: INTERNATIONAL BUSINESS MACHINES CORPORATION, ARMONK, NY (US)

(72) Inventors: Lowell Thomason, Byron, MN (US); Timothy Harold Brandt, Elysian, MN (US); John Rud, Rochester, MN (US); Kenneth Trisko, Stewartville, MN (US); Ronald Stanley Wesely, Rochester, MN (US)

(21) Appl. No.: 17/304,264

(22) Filed: Jun. 17, 2021

(51) Int. Cl. A61B 3/06; G06T 11/00; G06F 40/109; G06F 40/166

(52) U.S. Cl. CPC A61B 3/066; G06T 11/001; G06F 40/109; G06F 40/166

(57) ABSTRACT

Embodiments of the present invention analyze readable text in digital content displayed in a user interface for color deficiencies based on a user profile and determine a first color deficiency is present based on the user profile associated with a user viewing the readable text in the digital content. Embodiments of the present invention identify a first color substitution for the first color deficiency based on the user profile, where the user profile includes a color palette with a plurality of variations of a plurality of recognizable colors by the user and the first color substitution is from the plurality of variations of recognizable colors by the user. Embodiments of the present invention display the first color substitution for the first color deficiency as an overlay on the digital content, where the overlay covers at least the first color deficiency.

### BACKGROUND

This disclosure relates generally to color vision deficiency, and in particular to substituting text color based on color vision deficiency.

Color vision deficiency is an individual's reduced ability to view a color or differences between multiple colors, where a deficiency color is positioned between recognizable colors. Current digital content, such as emails and webpages, utilize a combination of font styles with varying foreground and background color, resulting in the individual having a reduced ability to view text in the digital content. For example, the individual might not be able to view red text with a green background but can view grey text on the green background. The ability of the user to view text in the digital content can be reduced to such a point where the individual might not even recognize that any text is present in the media content, resulting in the individual possibly missing critical information.

### SUMMARY

Embodiments in accordance with the present invention disclose a method, computer program product and computer system for color vision deficiency based text substitution, the method, computer program product and computer system can responsive to analyzing readable text in digital content displayed in a user interface for color deficiencies based on a user profile, determine a first color deficiency is present based on the user profile associated with a user viewing the readable text in the digital content. The method, computer program product and computer system can identify a first color substitution for the first color deficiency based on the user profile, wherein the user profile includes a color palette with a plurality of variations of a plurality of recognizable colors by the user and the first color substitution is from the plurality of variations of recognizable colors by the user. The method, computer program product and computer system can display the first color substitution for the first color deficiency as an overlay on the digital content, wherein the overlay covers at least the first color deficiency.

### BRIEF DESCRIPTION OF THE DRAWINGS

FIG. 1 is a functional block diagram illustrating a distributed data processing environment, in accordance with an embodiment of the present invention.

FIG. 2 is a flowchart depicting operational steps of a substitution program for generating a color palette for a user based on a color vision deficiency test, in accordance with an embodiment of the present invention.

FIG. 3 is a flowchart depicting operational steps of a substitution program for performing a color substitution in digital content based on the generated color palette for the user, in accordance with an embodiment of the present invention.

FIG. 4 is a block diagram of components of a computer system, such as the server computer of FIG. 1, in accordance with an embodiment of the present invention.

FIG. 5 depicts a cloud computing environment, in accordance with an embodiment of the present invention.

FIG. 6 depicts abstraction model layers, in accordance with an embodiment of the present invention.

### DETAILED DESCRIPTION

Embodiments of the present invention perform color substitutions for readable text in digital content, where the color substitutions relate to a foreground, a background, and font characteristics (e.g., shading). To determine color deficiencies for a user, the user performs a color vision deficiency test to determine which colors the user has difficulties viewing and to determine recognizable colors for the user. Subsequently, embodiments of the present invention generate a color palette based on the determined recognizable colors for performing a color substitution for readable text in digital content. Embodiments of the present invention can prompt the user with an option to customize the generated color palette and store any new selections from the user for future iterations. The generated color palette with the new selections from the user is stored as a user profile for the user, where the user profile is utilized when identifying a color substitution for a color deficiency. Embodiments of the present invention can analyze the readable text in the digital content and determine whether a color deficiency is present based on the user profile associated with the user viewing the digital content. Embodiments of the present invention identify a color substitution for the color deficiency based on the generated color palette of the user profile and displays the color substitution for the color deficiency as an overlay on the digital content. The user can provide feedback for the color substitution and embodiments of the present invention can utilize the feedback for the color substitution for future iterations.

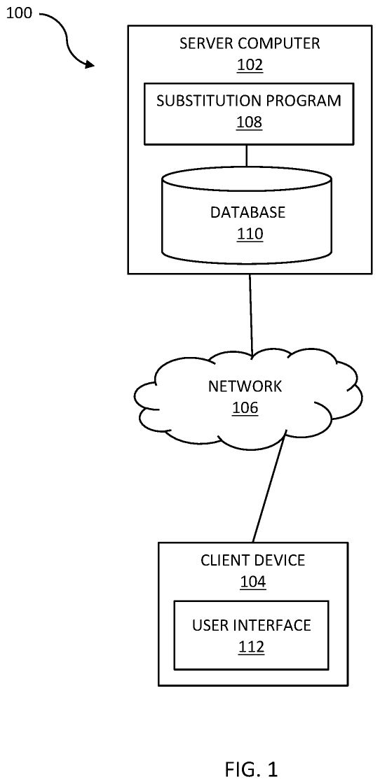

FIG. 1 is a functional block diagram illustrating a distributed data processing environment, generally designated 100, in accordance with one embodiment of the present invention. The term “distributed” as used herein describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

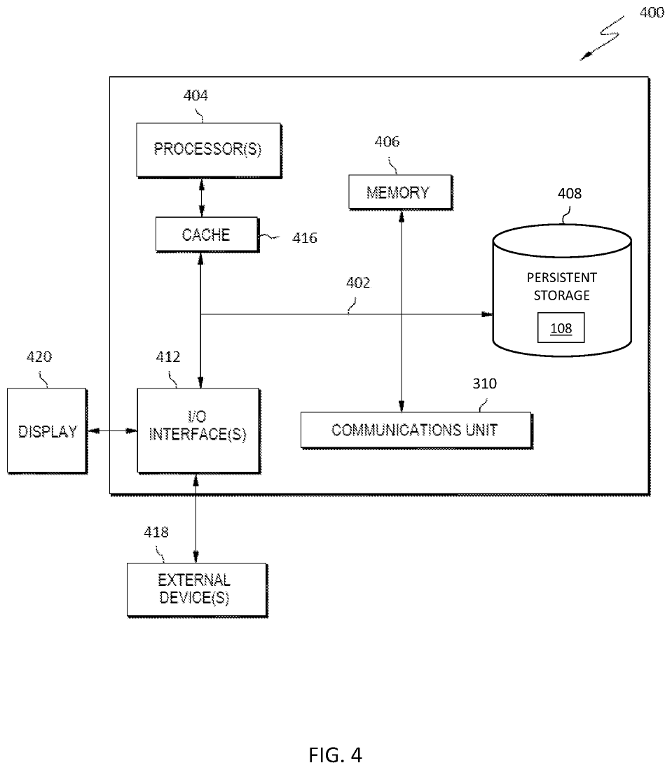

Distributed data processing environment includes server computer 102 and client device 104, interconnected over network 106. Server computer 102 can be a standalone computing device, a management server, a web server, a mobile computing device, or any other electronic device or computing system capable of receiving, sending, and processing data. In other embodiments, server computer 102 can represent a server computing system utilizing multiple computers as a server system, such as in a cloud computing environment. In another embodiment, server computer 102 can be a laptop computer, a tablet computer, a netbook computer, a personal computer (PC), a desktop computer, a smart phone, or any programmable electronic device capable of communicating with client device 104 and other computing devices (not shown) within the distributed data processing environment via network 106. In another embodiment, server computer 102 represents a computing system utilizing clustered computers and components (e.g., database server computers, application server computers, etc.) that act as a single pool of seamless resources when accessed within the distributed data processing environment. Server computer 102 includes substitution program 108 and database 110. Server computer 102 may include internal and external hardware components, as depicted and described in further detail with respect to FIG. 4.

Client device 104 can be a laptop computer, a tablet computer, a smart phone, smart watch, or any programmable electronic device capable of communicating with various components and devices within the distributed data processing environment, via network 106. Client device 104 may be a wearable computer, where wearable computers are miniature electronic devices that may be worn by the bearer under, with, or on top of clothing, as well as in or connected to glasses, hats, or other accessories. Wearable computers are especially useful for applications that require more complex computational support than merely hardware coded logics. In general, client device 104 represents one or more programmable electronic devices or combination of programmable electronic devices capable of executing machine readable program instructions and communicating with other computing devices (not shown) within the distributed data processing environment via a network, such as network 106. In one embodiment, client device 104 represents one or more devices associated with a user. Client device 104 includes an instance of user interface 112 for interacting with substitution program 108 on server computer 102.

Network 106 can be, for example, a telecommunications network, a local area network (LAN), a wide area network (WAN), such as the Internet, or a combination of the three, and can include wired, wireless, or fiber optic connections. Network 106 can include one or more wired and/or wireless networks capable of receiving and transmitting data, voice, and/or video signals, including multimedia signals that include voice, data, and video information. In general, network 106 can be any combination of connections and protocols that will support communications between server computer 102 and client device 104, and other computing devices (not shown) within the distributed data processing environment.

Substitution program 108 generates a color palette for a user based on a color vision deficiency test for a user. Substitution program 108 performs a color vision deficiency test and determines color deficiencies based on the color vision deficiency test for the user. Substitution program 108 also determines recognizable colors based on the color vision deficiency test for the user and stores the results as a user profile in database 110. Substitution program 108 generates a color palette based on the results with the recognizable colors and determines whether to customize the generated color palette based on user input. Responsive to substitution program 108 determining not to customize the generated color palette, substitution program 108 updates the user profile based on the generated color palette. Responsive to substitution program 108 determining to customize the generated color palette, substitution program 108 receives user color substitutions for the generated color palette. Substitution program 108 stores the user color substitutions for subsequent iterations and updates the user profile based on the generated color palette with the user color substitutions.

Substitution program 108 performs a color substitution in digital content based on the generated color palette for the user. Substitution program 108 identifies a user viewing digital content and analyzes readable text in the digital content for color deficiencies. Substitution program 108 determines a color deficiency is present based on the user profile associated with the user and identifies a color substitution for the color deficiency based on the user profile. Substitution program 108 displays the color substitution for the color deficiency as an overlay on the digital content and determines whether the user is providing color substitution feedback. Responsive to substitution program 108 determining the user is not providing color substitution feedback, substitution program 108 revert to analyzing the readable text in the digital content for color deficiencies. Responsive to substitution program 108 determining the user is providing color substitution feedback, receiving the color substitution feedback from the user, and updating the user profile based on the color substitution feedback.

Database 110 is a repository for data used by substitution program 108. In the depicted embodiment, database 110 resides on server computer 102. In another embodiment, database 110 may reside on client device 104 or elsewhere within the distributed data processing environment provided substitution program 108 has access to database 110. A database is an organized collection of data. Database 110 can be implemented with any type of storage device capable of storing data and configuration files that can be accessed and utilized by substitution program 108, such as a database server, a hard disk drive, or a flash memory. Database 110 stores data used by substitution program 108, such as, user profiles, color vision deficiency test, color deficiency results, determined color deficiencies for each user, determined recognizable colors for each user, generated color palettes, color substitutions for the color palettes, and the like.

User interface 112 enables a user to make requests of or issue commands to client device 104 and receive information and instructions in response. In one embodiment, a user of client device 104 accesses user interface 112 via voice commands in natural language. In one embodiment, user interface 112 may be a graphical user interface (GUI) or a web user interface (WUI) and can display text, documents, web browser windows, user options, application interfaces, and instructions for operation, and include the information (such as graphic, text, and sound) that a program presents to a user and the control sequences the user employs to control the program. In another embodiment, user interface 112 may also be mobile application software. In an example, mobile application software, or an “app,” is a computer program designed to run on smart phones, tablet computers and other mobile devices. User interface 112 enables a user of client device 104 to interact with substitution program 108 and view digital content with an overlay with the color substitutions for the color deficiency.

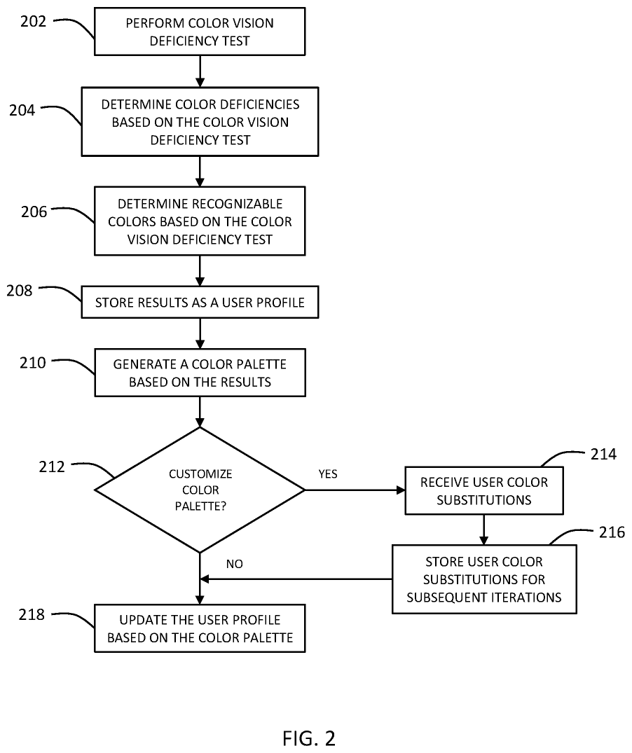

FIG. 2 is a flowchart depicting operational steps of a substitution program for generating a color palette for a user based on a color vision deficiency test, in accordance with an embodiment of the present invention.

Substitution program 108 performs a color deficiency test (202). In this embodiment, substitution program 108 utilizes a color vision deficiency test with responses received from a user to determine color deficiencies and recognizable colors for the user. For a client device associated with the user (e.g., mobile phone, smart TV, tablet computer), substitution program 108 temporarily defaults color display settings for the client device associated with the user and displays the color deficiency test in a user interface on the client device. Substitution program 108 displays the color deficiency test utilizing the default color display settings to ensure the results are not skewed based on previously altered setting by the user and/or another person without the user's knowledge. Substitution program 108 can store one or more color vision deficiency tests in a database, along with results from the color vision deficiency tests for each user of substitution program 108. Alternatively, substitution program 108 can access an online based color vision deficiency test from a public provider and display the online based color vision deficiency test in the user interface on the client device with the default color display settings. Subsequent to the user completing the color deficiency test, substitution program 108 can analyze the results and/or receive the results directly from the public provider.

Substitution program 108 determines color deficiencies based on the color vision deficiency test (204). Color deficiency is defined as a reduced ability to view a color or differences between multiple colors, where a deficiency color is positioned between recognizable colors. Substitution program 108 determines color deficiencies for the user based on results from the color vision deficiency test. In one example, substitution program 108 determines the user was unable to detect letters, characters, and/or numbers that were projected during the color vision deficiency test, where the letters, characters, and/or numbers undetected by the user were various shades of green and blue. Based on the color vision deficiency test, substitution program 108 determines color deficiencies in shades of green and blue for the colors on the visible spectrum for the user. Furthermore, substitution program 108 determines the user had difficultly detecting letters, characters, and/or numbers that were projected during the color vision deficiency test based on a response time being above a threshold level (e.g., 5 seconds). The letters, characters and/or number for which the user had difficulty detecting included shades of red on shades of green background. Based on the color vision deficiency test, substitution program 108 determined color deficiencies in shades of red when paired with shades of green background, where the shades of red are considered foreground colors (i.e., letters, characters, and/or numbers).

Substitution program 108 determines recognizable colors based on the color vision deficiency test (206). In this embodiment, the recognizable colors represent portions of the visible spectrum viewable by the user based on the results of the color vision deficiency test. The portions of the visible spectrum viewable by the user exclude the determined color deficiencies in (204). Furthermore, substitution program 108 also determines recognizable color combinations that include a first color for a foreground and a second color for a background viewable by the user, where the first color is assigned to the various the letters, characters, and/or numbers. The determined recognizable color combinations viewable by the user exclude the determined color deficiency combinations in (204). Continuing from the example above, substitution program 108 determines the recognizable colors for the user to include all shades of colors that are not green and blue, since substitution program 108 previously determined the user to have color deficiency when viewing letters, characters, and/or numbers in shades of green and blue. Furthermore, substitution program 108 determines the recognizable color combinations excluding the paired color deficiency of red with green background and any recognizable color combination that includes shades of green and blue. In some embodiments, substitution program 108 can rank the recognizable colors from most recognizable to least recognizable based on a response time measured for detecting letters, characters, and/or numbers during the color vision deficiency test, where most recognizable colors would have a faster response time compared to lesser recognizable colors with slower response times.

Substitution program 108 stores results as a user profile (208). Substitution program 108 stores the results of the color vision deficiency test, along with the determined color deficiencies and the determined recognizable colors for the user as a user profile. In the event a user profile does not exist for the user, substitution program 108 queries the user to create a profile for which the results of the color vision deficiency test are applied. In the event a user profile exists for the user, substitution program 108 updates the user profile with the current color vision deficiency test and updates the determined color deficiencies and the determined recognizable colors for the user, since they may have changed since the previous color vision deficiency test. Each user profile can include information for one or more client devices associated with the user, where substitution program 108 performs a color substitution in digital content displayed on the one or more client devices associated with the user. Furthermore, substitution program 108 utilizes information for the one or more client device to determine when the user is near another device that is not associated with the user profile, where substitution program 108 can perform a color substitution in digital content on the other device when substitution program 108 detects that the user is near the other device.

Substitution program 108 generates a color palette based on the results (210). Substitution program 108 generates a color palette based on the results that includes the recognizable colors. Substitution program 108 generates a predetermined number (e.g., five) of variations (i.e., shades) for each color from the recognizable colors to populate the color palette for the user who performed the color vision deficiency test. Substitution program 108 also generates a predetermined number (e.g., three) of variations for each color from the recognizable color combinations viewable by the user who performed the color vision deficiency test. Substitution program 108 can reduce the populated color variations of the color palette based on the ranking of the recognizable colors from most recognizable to least recognizable, as discussed above with regards to (206). Substitution program 108 can reduce the populated color variations of the color palette by removing the least recognizable colors (e.g., bottom 25%) by the user who performed the color vision deficiency test. Substitution program 108 generates a color palette in two portions, a first portion includes subset of color variations for each recognizable colors and a second portion includes a subset of color variations for each color from the recognizable color combinations.

Substitution program 108 determines whether to customize the generated color palette based on user input (decision 212). Substitution program 108 can query the user via a user interface on a client device associated with the user to determine whether to customize the generated color palette. Substitution program 108 can display the generated color palette in the user interface on the client device and display a user selectable option to customize the generated color palette via one or more color substitutions by the user. In the event substitution program 108 determines to customize the generated color palette (“yes” branch, decision 212), substitution program 108 receives user color substitutions for the generated color palette (214). In the event substitution program 108 determines not to customize the generated color palette (“no” branch, decision 212), substitution program 108 updates the user profile based on the generated color palette (218).

Substitution program 108 receives user color substitutions for the generated color palette (214). For discussion purposes, color substitutions can include replacing, adding, or removing one or more color variations for each recognizable color and/or recognizable color combinations. Substitution program 108 displays the generated color palette, where the user can select each color variation of each recognizable color or recognizable color combination to replace, add, or remove. In one example, substitution program 108 displays the generated color palette with five variations for each recognizable color, where the user has difficulty viewing three of the five variations for a recognizable color (e.g., purple). Substitution program 108 receives a user color substitution that includes removing the three of the five variations for the recognizable color from the generated color palette. Alternatively, substitution program 108 receives a user color substitution that include adjusting a shade of each of the three variations to improve viewability and replacing the three variations for the recognizable color from the generated color palette.

In another example, substitution program 108 displays the generated color palette with three variations for each recognizable color combination, where the user has difficulty viewing two of the three variations for a specific recognizable color combination. A first variation for the specific recognizable color combination includes a red foreground with blue background, a second variation includes a red foreground with a white background, and a third variation include a red foreground with a black background. The user has difficulty viewing the first variation and the third variation and as a result, substitution program 108 receives a user color substitution that includes removing the first variation and the second variation. Additionally, substitution program 108 receives a user color substitution that includes adding a fourth variation with a red foreground and light gray background.

Substitution program 108 stores the user color substitution for subsequent iterations (216) and updates the user profile based on the generated color palette (218). Substitution program 108 stores the user color substitutions along with the user profile for the user for subsequent iterations of substitution program 108 utilizing the generated color palette to perform color substitutions on digital content. Substitution program 108 utilizes iterative machine learning through feedback from the user to determine which color substitutions are preferred by the user for each type of digital content and rank the various variations for each of the recognizable color and recognizable color combinations. Furthermore, Substitution program 108 utilizes iterative machine learning through feedback from the user to determine which color substitutions are preferred by the user for each device that the user is utilizing to view digital content. Substitution program 108 stores the preferences for each device for future color substitutions on digital content displayed on each of the devices.

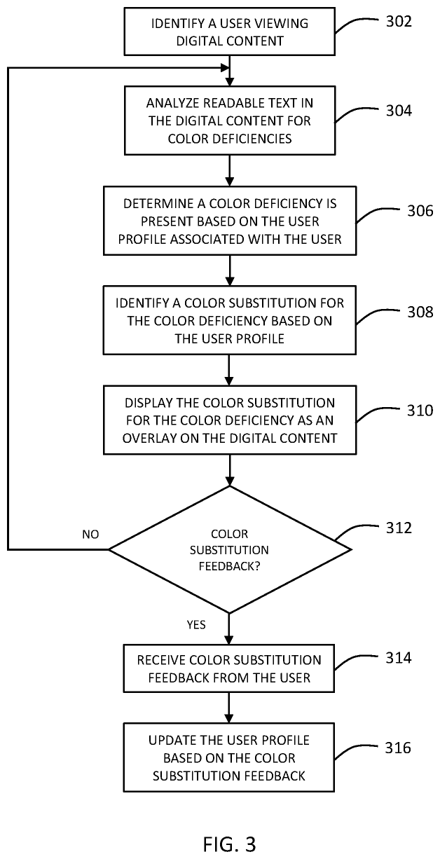

FIG. 3 is a flowchart depicting operational steps of a substitution program for performing a color substitution in digital content based on the generated color palette for the user, in accordance with an embodiment of the present invention.

Substitution program 108 identifies a user viewing digital content (302). In this embodiment, substitution program 108 identifies a user viewing digital content based on a user profile being utilized on a client device, where the user is viewing the digital content in a user interface on the client device. The client device can utilize multiple profiles, where substitution program 108 performs color substitutions based on the multiple profiles for the client device. If a user profile is not implemented on the client device, substitution program 108 queries the user to select a user profile from the multiple profiles to perform color substitutions based on the selected user profile. In another embodiment, the user with a client device approaches another electronic device (e.g., smart tv, tablet computer) capable of displaying digital content, where the user does not have an established user profile on the other electronic device. In response to substitution program 108 determining that a user interface and display settings on the other electronic device is accessible via a network connection, substitution program 108 determines the user is viewing digital content on the other electronic device and identifies the user viewing digital content based on a user profile on the client device. Substitution program 108 performs color substitutions on the other electronic device utilizing the user profile on the client device.

Substitution program 108 analyzes readable text in the digital content for color deficiencies (304). Substitution program 108 analyzes readable text in the digital content for color deficiencies by identifying letters, characters, and/or numbers present in the digital content utilizing image detection software and/or optical character recognition. Substitution program 108 can identify letters, characters, and/or numbers in media articles, emails, messages, forum posts, blog posts, subtitles, captions, and the like that are present in the digital content. Substitution program 108 can utilize optical character recognition to identify each instance of a letter, character, and/or number present in the readable text in the digital text, whether it be typed, scanned, or handwritten. Substitution program 108 can create block around each letter, character, and/or number that shares a common foreground color or foreground and background color combination. Substitution program 108 can utilize these blocks to display an overlay in each block to ensure the color substitutions are restricted to the block and not overlayed on the entire digital content present in the user interface viewable by the user.

Substitution program 108 determines a color deficiency is present based on the user profile associated with the user (306). Substitution program 108 identifies a foreground color for each of the letters, characters, and/or numbers and a background color for each of the letters, characters, and/or numbers. Substitution program 108 compares the identified foreground color to the determined color deficiency (e.g., shades of blue and green) for the identified user based on the user profile and determines if a color deficiency is present. Substitution program 108 also compares the identified foreground and background color to the determined color deficiency associated with paired colors (e.g., red paired with shades of green background) for the identified user based on the user profile and determines if a color deficiency is present. If the foreground color and/or a combination of the foreground and background color are present in the readable text in the digital content, substitution program 108 determines a color deficiency is present in the readable text in the digital content.

Substitution program 108 identifies a color substitution for the color deficiency based on the user profile (308). Substitution program 108 utilizes the stored color palette associated with the user profile to identify a color substitution for the foreground (i.e., letters, characters, and/or numbers) or the combined foreground and background that include the color deficiency. As previously discussed in FIG. 2, the color palette includes various user recognizable colors that are utilized by substitution program 108 to perform a color substitution for readable text with the color deficiency that is not recognizable or difficult for the user to recognize. In one example, substitution program 108 identified readable text that is displayed in a shade of green in a user interface on a client device, where the user has a color deficiency when viewing shades of green. Substitution program 108 identifies a color substitution for the color deficiency utilizing that color palette, where substitution program 108 identifies dark grey as a color substitution for instances where shades of green are utilized for the readable text. In another example, substitution program 108 identified readable text that is displayed in red with a green background, where the user has a color deficiency when viewing a red foreground with a background in a shade of green. Substitution program 108 identifies a color substitution for the color deficiency utilizing that color palette, where substitution program 108 identifies a white background as a color substitution for the color deficiency when a red foreground is paired with a green background.

Substitution program 108 displays the color substitution for the color deficiency as an overlay on the digital content (310). Since substitution program 108 may not be able to alter the readable text in the digital content, substitution program 108 utilizes a localized overlay on the digital content for displaying the color substitution for the color deficiency, wherein the localized overlays covers at least the color deficiency. As discussed above with regards to (304), substitution program 108 can utilize multiple blocks to display an overlay in each of blocks to ensure the color substitutions are restricted to each block and not overlayed on the entire digital content present in the user interface viewable by the user. In one example, the digital content includes the sentence, “Version 1.2B is expected to be released to the public on October 1^{st}” where “October 1^{st}” includes a foreground color that is a determined color deficiency (e.g. green) based on the user profile. Substitution program 108 utilizes a block around “October 1^{st}” to alter the foreground (i.e., letters and numbers) or background color within the block utilizing the overlay. As a result, substitution program 108 displays the color substitution for the color deficiency as an overlay on the digital content in the block with the portion, “October 1^{st}”. In another embodiment, substitution program 108 utilizes optical character recognition to analyze the readable text in the digital content and can utilizes optical character recognition to determine where to display the color substitution for the color deficiency as an overlay, such that the overlay covers each letter, characters, and/or number.

Substitution program 108 determines whether the user is providing color substitution feedback (decision 312). Substitution program 108 can query the user via a user interface on a client device associated with the user for feedback regarding the color substitution. The query can include options for selecting whether the color substitution improved viewability of the readable text in the digital content or worsened viewability of the readable text. Furthermore, substitution program 108 can include another option allowing for the user to manual override the identified color substitution by substitution program 108, where substitution program 108 can receive a manual selection for a color substitution for the color deficiency. In the event substitution program 108 determines the user is providing color substitution feedback, (“yes” branch, decision 312), substitution program 108 receives the color substitution feedback from the user (314). In the event substitution program 108 determines the user is not providing color substitution feedback (“no” branch, decision 312, substitution program 108 reverts to analyzing the readable text in the digital content for color deficiencies (304).

Substitution program 108 receives the color substitution feedback from the user (314). Substitution program 108 utilizes the feedback from the user to identify patterns (i.e., machine learning) for the user in determining color substitution based on color deficiency, color combination deficiencies, and/or types of digital content being viewed by the user. In one embodiment, substitution program 108 receives a feedback selection from the user indicating that the color substitution improved viewability of the readable text in the digital content. Substitution program 108 utilizes this feedback for future iterations of similar color deficiencies (e.g., shades of green and blue) and/or of similar digital content (e.g., emails displayed in program A), by utilizing the color substitution performed in the present iteration for the future iterations. In another embodiment, substitution program 108 receives a feedback selection from the user indicating that the color substitution worsened viewability of the readable text in the digital content. Substitution program 108 utilizes this feedback for future iterations of similar color deficiencies and/or of similar digital content, by avoiding the color substitution performed in the present iteration for the future iterations. In yet another embodiment, substitution program 108 receives a feedback selection from the user that includes a manual color substitution. Substitution program 108 utilizes this feedback for future iterations of similar color deficiencies and/or of similar digital content, by utilizing the manual color substitution for future iterations of similar color deficiencies and/or similar digital content.

Substitution program 108 updates the user profile based on the color substitution feedback (316). Substitution program 108 updates the user profile for the user based on the color substitution feedback for the future iterations. With the updates, substitution program 108 alters the color palette with the recognizable colors to ensure the color palette for the color substitution aligns with the feedback being received from the user. Substitution program 108 can replace, ad, or remove one or more color variations for each recognizable color and/or recognizable color combinations based on the color substitution feedback being received from the user.

FIG. 4 depicts computer system 400, where server computer 102 is an example of a computer system 400 that includes substitution program 108, respectively. The computer system includes processors 404, cache 416, memory 406, persistent storage 408, communications unit 410, input/output (I/O) interface(s) 412 and communications fabric 402. Communications fabric 402 provides communications between cache 416, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a crossbar switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of processors 404 by holding recently accessed data, and data near recently accessed data, from memory 406.

Program instructions and data used to practice embodiments of the present invention may be stored in persistent storage 408 and in memory 406 for execution by one or more of the respective processors 404 via cache 416. In an embodiment, persistent storage 408 includes a magnetic hard disk drive. Alternatively, or in addition to a magnetic hard disk drive, persistent storage 408 can include a solid state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices. In these examples, communications unit 410 includes one or more network interface cards. Communications unit 410 may provide communications through the use of either or both physical and wireless communications links. Program instructions and data used to practice embodiments of the present invention may be downloaded to persistent storage 408 through communications unit 410.

I/O interface(s) 412 allows for input and output of data with other devices that may be connected to each computer system. For example, I/O interface 412 may provide a connection to external devices 418 such as a keyboard, keypad, a touch screen, and/or some other suitable input device. External devices 418 can also include portable computer readable storage media such as, for example, thumb drives, portable optical or magnetic disks, and memory cards. Software and data used to practice embodiments of the present invention can be stored on such portable computer readable storage media and can be loaded onto persistent storage 408 via I/O interface(s) 412. I/O interface(s) 412 also connect to display 420.

Display 420 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

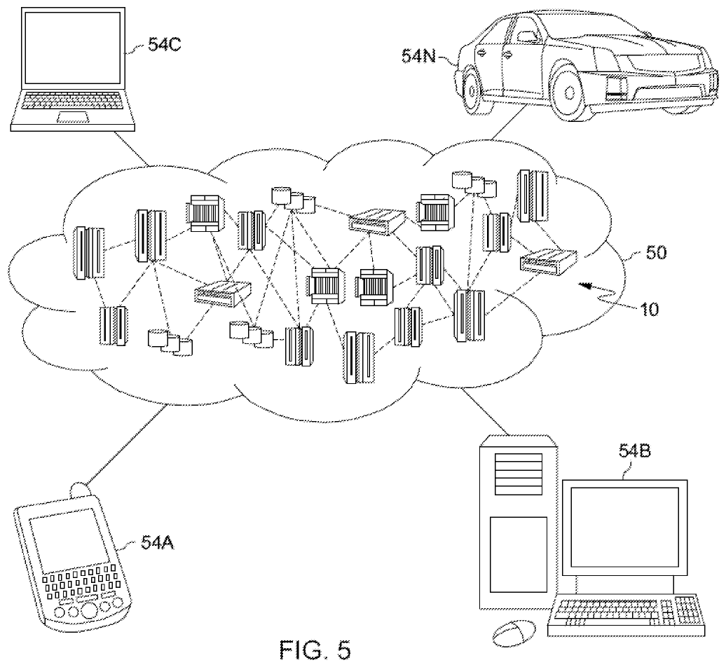

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

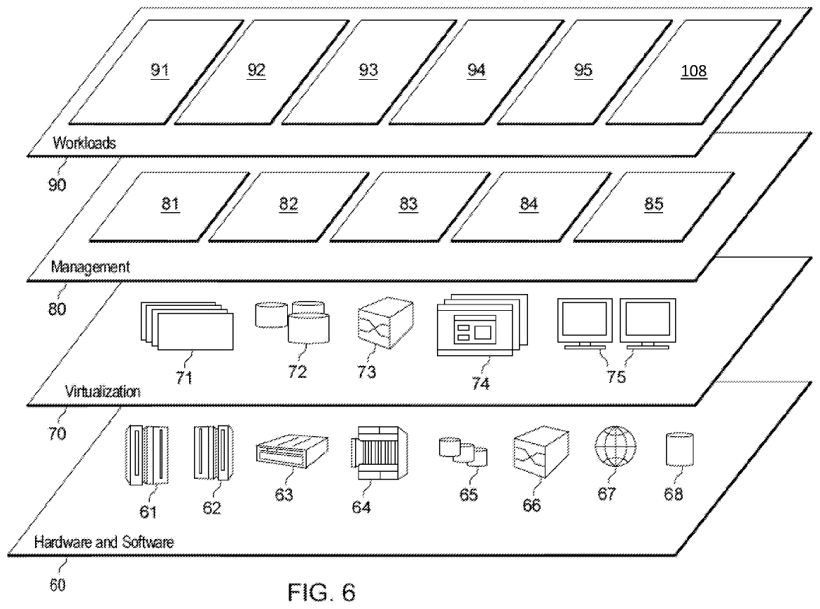

Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 include hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and substitution program 108.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method comprising:
responsive to analyzing readable text in digital content displayed in a user interface for color deficiencies based on a user profile, determining a first color deficiency is present based on the user profile associated with a user viewing the readable text in the digital content;
identifying a first color substitution for the first color deficiency based on the user profile, wherein the user profile includes a color palette with a plurality of variations of a plurality of recognizable colors by the user and the first color substitution is from the plurality of variations of recognizable colors by the user; and
displaying the first color substitution for the first color deficiency as an overlay on the digital content, wherein the overlay covers at least the first color deficiency.

2. The computer-implemented method of claim 1, further comprising:
identifying the user viewing the digital content based on the user profile associated with a client device with the user interface.

3. The computer-implemented method of claim 1, further comprising:
responsive to determining the user is providing color substitution feedback, receiving a first color substitution feedback from the user indicating the first color substitution improved viewability of the readable text associated with the first color deficiency; and
updating the user profile based on the first color substitution feedback for future color substitutions.

4. The computer-implemented method of claim 1, further comprising:
responsive to determining the user is providing color substitution feedback, receiving a first color substitution feedback from the user indicating the first color substitution worsened viewability of the readable text associated with the first color deficiency; and
updating the user profile based on the first color substitution feedback for future color substitutions.

5. The computer-implemented method of claim 1, further comprising:
responsive to the user performing a color vision deficiency test, determining one or more color deficiencies for the user based on the color vision deficiency test, wherein the one or more color deficiencies includes the first color deficiency;
determining the plurality of recognizable colors by the user based on the color vision deficiency test; and
storing results from the color vision deficiency test as the user profile, wherein the results include the one or more color deficiencies for the user and the plurality of recognizable colors by the user.

6. The computer-implemented method of claim 5, further comprising:
generating the color palette with the plurality of variations of the plurality of recognizable colors by the user, wherein each recognizable color from the plurality of recognizable colors includes a subset of variations from the plurality of variations.

7. The computer-implemented method of claim 6, further comprising:
responsive to determining to customize the color palette, receiving a user color substitution for the color palette;
storing the user color substitution for the future color substitutions; and
updating the user profile based on the color palette with the user color substitution.

8. A computer program product comprising one or more computer readable storage media and program instructions collectively stored on the one or more computer readable storage media, the program instructions executable by one or more processors to cause the one or more processors to perform a method comprising:
responsive to analyzing readable text in digital content displayed in a user interface for color deficiencies based on a user profile, determining a first color deficiency is present based on the user profile associated with a user viewing the readable text in the digital content;
identifying a first color substitution for the first color deficiency based on the user profile, wherein the user profile includes a color palette with a plurality of variations of a plurality of recognizable colors by the user and the first color substitution is from the plurality of variations of recognizable colors by the user; and
displaying the first color substitution for the first color deficiency as an overlay on the digital content, wherein the overlay covers at least the first color deficiency.

9. The computer program product of claim 8, the method further comprising:
identifying the user viewing the digital content based on the user profile associated with a client device with the user interface.

10. The computer program product of claim 8, the method further comprising:
responsive to determining the user is providing color substitution feedback, receiving a first color substitution feedback from the user indicating the first color substitution improved viewability of the readable text associated with the first color deficiency; and
updating the user profile based on the first color substitution feedback for future color substitutions.

11. The computer program product of claim 8, the method further comprising:
responsive to determining the user is providing color substitution feedback, receiving a first color substitution feedback from the user indicating the first color substitution worsened viewability of the readable text associated with the first color deficiency; and
updating the user profile based on the first color substitution feedback for future color substitutions.

12. The computer program product of claim 8, the method further comprising:
responsive to the user performing a color vision deficiency test, determining one or more color deficiencies for the user based on the color vision deficiency test, wherein the one or more color deficiencies includes the first color deficiency;
determining the plurality of recognizable colors by the user based on the color vision deficiency test; and
storing results from the color vision deficiency test as the user profile, wherein the results include the one or more color deficiencies for the user and the plurality of recognizable colors by the user.

13. The computer program product of claim 12, the method further comprising:
generating the color palette with the plurality of variations of the plurality of recognizable colors by the user, wherein each recognizable color from the plurality of recognizable colors includes a subset of variations from the plurality of variations.

14. The computer program product of claim 13, the method further comprising:
responsive to determining to customize the color palette, receiving a user color substitution for the color palette;
storing the user color substitution for the future color substitutions; and
updating the user profile based on the color palette with the user color substitution.

15. A computer system comprising:
one or more processors; and
one or more computer readable storage media, wherein the one or more processors are structured, located, connected and/or programmed to execute program instructions collectively stored on the one or more computer readable storage media; and
the program instructions, when executed by the one or more processors, cause the one or more processors to perform a method comprising:
responsive to analyzing readable text in digital content displayed in a user interface for color deficiencies based on a user profile, determining a first color deficiency is present based on the user profile associated with a user viewing the readable text in the digital content;
identifying a first color substitution for the first color deficiency based on the user profile, wherein the user profile includes a color palette with a plurality of variations of a plurality of recognizable colors by the user and the first color substitution is from the plurality of variations of recognizable colors by the user; and
displaying the first color substitution for the first color deficiency as an overlay on the digital content, wherein the overlay covers at least the first color deficiency.

16. The computer system of claim 15, the method further comprising:
identifying the user viewing the digital content based on the user profile associated with a client device with the user interface.

17. The computer system of claim 15, the method further comprising:
responsive to determining the user is providing color substitution feedback, receiving a first color substitution feedback from the user indicating the first color substitution improved viewability of the readable text associated with the first color deficiency; and
updating the user profile based on the first color substitution feedback for future color substitutions.

18. The computer system of claim 15, the method further comprising:
responsive to determining the user is providing color substitution feedback, receiving a first color substitution feedback from the user indicating the first color substitution worsened viewability of the readable text associated with the first color deficiency; and
updating the user profile based on the first color substitution feedback for future color substitutions.

19. The computer system of claim 15, the method further comprising:
responsive to the user performing a color vision deficiency test, determining one or more color deficiencies for the user based on the color vision deficiency test, wherein the one or more color deficiencies includes the first color deficiency;
determining the plurality of recognizable colors by the user based on the color vision deficiency test; and
storing results from the color vision deficiency test as the user profile, wherein the results include the one or more color deficiencies for the user and the plurality of recognizable colors by the user.

20. The computer system of claim 19, the method further comprising:
generating the color palette with the plurality of variations of the plurality of recognizable colors by the user, wherein each recognizable color from the plurality of recognizable colors includes a subset of variations from the plurality of variations.
